(19) United States
(12) Patent Application Publication
(10) Pub. No.: US 2026/0248810 A1
(43) Pub. Date: Aug. 27, 2026

(54) COMPOSITIONS FOR TREATING A SUBJECT FROM SEXUAL DYSFUNCTION

(71) Applicant: Kim VAN ROOIJ, Amstelveen (NL)

(72) Inventor: Kim VAN ROOIJ, Amstelveen (NL)

(21) Appl. No.: 19/489,648

(22) PCT Filed: Jun. 6, 2024
(86) PCT No.: PCT/NL2024/050294

§ 371 Date: Dec. 3, 2025

(30) Foreign Application Priority Data
Jun. 6, 2023 (NL) 2035009

(51) Int. Cl. A61K 31/568; A61K 9/00; A61K 38/095; A61P 15/00; A61P 15/10

(52) U.S. Cl. CPC A61K 31/568; A61K 9/006; A61K 38/095; A61P 15/00; A61P 15/10

(57) ABSTRACT

The invention relates to steroids and/or functional analogues thereof for use in a method of treating an individual suffering from sexual dysfunction. Said steroids may be combined with an effective dosage of oxytocin and/or a functional analogue thereof.

FIELD: The invention relates to the field of drug formulation and drug delivery. More specifically, the invention relates to a combination of active ingredients for administration to subjects suffering from sexual dysfunction.

### BACKGROUND OF THE INVENTION

The main symptoms of the diagnosis sexual dysfunction or “Sexual Interest/Arousal Disorder (SIAD)” are low sexual desire and/or arousal (American Psychiatric Association. Diagnostic and Statistical Manual of Mental Disorders. 2013). This condition negatively influences psychological well-being and results in personal distress (Davison et al., 2009. J Sex Med 6:2690-2697). Although SIAD is a common condition which is prevalent among individuals of all ages and ethnicities, little is known about its etiology.

Attempts to develop a drug treatment for treatment of sexual dysfunction have been guided by the principle of “one size fits all”, and have failed to acknowledge the complexity of sexuality. Sex and its problems form an integral part of human existence, evoking strong emotional responses and fierce debates among scientists and in the public domain. Each message in the media about drug development for treatment of sexual dysfunction raises a schism: It becomes nurture against nature (and vice versa), seldom the combination of the two. People tend to acclimate to sexual experiences and adjust their expectations accordingly. Thus, by the time they arrive at a clinician's office, they have lived with low desire and/or arousal for a relatively long time. Biological constitution forms a foundation upon which experiences either raise or diminish expectations. For example, in women with Female Sexual Interest/Arousal Disorder (FSIAD), two biological constraints that underlie the interpretation of their own sexual desire and arousal can be distinguished: 1) a relatively insensitive system for sexual cues, and 2) dysfunctional activation of sexual inhibitory mechanisms.

Similar biological constraints may also underly male sexual dysfunction, which are often not recognized as such and typed under the name ‘erectile dysfunction’.

Treatment of sexual dysfunction may be provided by testosterone or a phosphodiesterase type 5 inhibitor (PDE-51) for males; and by testosterone combined with a PDE-5i, or testosterone combined with a 5-hydroxytryptamine Receptor 1A receptor agonist (5HT1A-ra), for females.

However, some males, estimated to be around 30-40%, do not respond at either testosterone, or a PDE-5i such as Viagra. In the above suggested treatments for women (in development), it is unclear which treatment is most suitable for which patient.

There is thus a need to provide further, and/or, more effective treatment options for individuals from sexual dysfunction.

### BRIEF DESCRIPTION OF THE INVENTION

The invention therefore provides for further and/or improved treatments of sexual dysfunction. Advantageously, in the invention testosterone and oxytocin (and, likewise, functional analogues thereof), have the potential to reinforce each other, i.e. testosterone increases sexual motivation and oxytocin enhances the feeling of partner bonding. Without being bound by theory, highly advantageously, the different mechanisms of actions combined can bridge the distinction in two previously distinguished subgroups in sexual dysfunctions (low sensitivity to sexual stimuli/cues vs. high activity of sexual inhibitory mechanisms).

The invention therefore provides testosterone and/or a functional analogue thereof for use in a treatment of a subject suffering from sexual dysfunction, the treatment comprising administering an effective dosage of testosterone and/or a functional analogue (for example dihydrotestosterone) thereof to thereby treat said subject suffering from sexual dysfunction. Furthermore, and highly preferably, the treatment further comprises administering an effective dosage of oxytocin and/or a functional analogue thereof.

For example, in the art sublingual formulations are known and available for administering effective dosages of testosterone and/or a functional analogue thereof, and for oxytocin intranasal formulations are known and available for administering effective dosages of oxytocin and/or a functional analogue thereof. Of course, the invention may not be restricted to these types of formulations, but at least these routes of administration are highly convenient in the context of treatment of sexual dysfunction and allow for on-demand personal use thereof in a private setting.

### FIGURES

FIG. 1. Plotted in this graph is measured plasma levels of total testosterone (mmol/L, left graph) and plasma levels of free testosterone (pmol/L, right graph), prior to administration (left bar, before), after testosterone administration (middle bar, T) and after administration of a combination of testosterone and dihydrotestosterone (right bar, T+DHT). The amount of free testosterone is higher with the combination of testosterone and dihydrotestosterone as compared with testosterone alone.

FIG. 2. Plotted in this graph is sexual arousal preceding sexual activity (left) and during sexual activity (right) upon treatment with a combination of testosterone and Viagra (left bar) and upon treatment with a combination of testosterone, dihydrotestosterone and Viagra (right bar). The treatment comprising both testosterone and dihydrotestosterone resulted in the highest arousal.

FIG. 3. Plotted in this graph is sexual performance, comparing no-drug (left bars) and treatment with a combination of testosterone and oxytocin (right bars), with from left to right scores for sexual arousal during sexual activity, orgasm, minutes before orgasm, horniness preceding sexual activity and horniness during sexual activity. The scores for sexual arousal during sexual activity, orgasm, preceding sexual activity and horniness during sexual activity showed an increase for the combination of testosterone and oxytocin, while the score for minutes before orgasm showed a reduction.

### DEFINITIONS

The term “sexual dysfunction”, as is used herein, refers to an individual that experiences difficulties during any stage of normal sexual activity, including physical pleasure, desire, preference, arousal, or orgasm. The World Health Organization (WHO) defines sexual dysfunction as a “person's inability to participate in a sexual relationship as they would wish” (WHO. International Statistical Classification of Diseases and Related Health Problems, 10th Revision (ICD-10). Geneva, Switzerland: WHO; 2010). Said individual often feels extreme distress and interpersonal strain for a minimum of six months. Sexual dysfunction can have a profound impact on an individual's perceived quality of sexual life. The term may include male sexual dysfunction, such as Hypoactive Sexual Desire Disorder (HSDD), erectile dysfunction, premature ejaculation, delayed ejaculation and anorgasmia. In female sexual dysfunction symptoms may be: dyspareunia, vaginismus, anorgasmia and Female Sexual Interest/Arousal Disorder (FSIAD).

The term Sexual Interest/Arousal Disorder (SIAD), as is used herein, refers to a loss of desire to sexual triggers/cues that an individual previously experienced as arousing. An individual suffering from SIAD will experience these symptoms of low or absent sexual interest/arousal as distressing. FSIAD is defined in the DSM-5 as lack of, or significantly reduced, sexual interest/arousal.

The term “sublingual formulation”, as is used herein, refers to a formulation of an active ingredient that is suitable for administration under the tongue, also termed hypoglossal or subglossal. A sublingual formulation allows the release of an active ingredient, in the present case a testosterone and/or a functional analogue thereof, when held in the oral cavity such as under the tongue, preferably within minutes, and allows the active ingredient to diffuse through the mucous membrane into the blood. Administration of an active ingredient by a sublingual formulation therefore bypasses the gastro-intestinal tract.

The term “sublingual”, also termed “buccal”, as is used herein, refers to the interspace between the lips and the teeth and the interspace between the cheek and the teeth. The sublingual area is delimited by the palate and tongue and includes the true sublingual area.

The term “cyclodextrin”, as is used herein, includes reference to a displacement enhancer such as hydroxypropylbeta-cyclodextrin, poly-betacyclodextrin or gamma-cyclodextrin.

The terms “treating” and “treatment”, as are used herein, refer to reduction in severity and/or frequency of symptoms, elimination of symptoms and/or underlying cause, retardation of progression of a condition such as sexual dysfunction. Thus, for example, “treating” a human involves amelioration of symptoms and treatment of a clinically symptomatic human by inhibiting or causing regression of a condition such as sexual dysfunction. The therapeutic effect preferably is a partial or complete cure of said condition.

The term “free testosterone”, as is used herein, refers to a short, sharp peak level of free testosterone of at least 0,010 nmol/l in the blood circulation of the human. A testosterone in the circulation is typically bound by steroid hormone binding globulin (SHBG) and by albumin. It is important that the peak plasma level of testosterone is present and calculated as free testosterone, so a fraction not bound by albumin and SHBG. Hence, the dose of testosterone that is provided should be high enough to saturate the albumin and SHBG (i.e. the concentration of testosterone and/or functional analogue thereof must be high enough to overcome direct, complete binding of testosterone by SHBG or albumin), or another way of avoiding binding to albumin or SHBG must be designed, such as the use of a competitor for the testosterone binding site on SHBG.

### DETAILED DESCRIPTION OF THE INVENTION

The invention provides for testosterone and/or a functional analogue thereof for use in a treatment of a subject suffering from sexual dysfunction, the treatment comprising administering an effective dosage of testosterone and/or a functional analogue thereof to thereby treat said subject suffering from sexual dysfunction.

Testosterone is also known under the chemical name 17-β-hydroxyandrost-4-en-3-one. Testosterone can be obtained in various ways: it may be isolated and purified from nature or synthetically produced by any manner. The term “functional analogue of testosterone” as used herein relates to any useful metabolite or precursor of testosterone, for example the metabolite dihydrotestosterone that can provide the same function as testosterone. It is clear to the skilled person that if a metabolite or precursor of testosterone is used, i.e. a functional analogue of testosterone, the time point for administration may be appropriately adjusted if needed, relative to a selected time point of administration of testosterone.

The invention further provides for testosterone and/or a functional analogue thereof for use in a treatment of a subject suffering from sexual dysfunction, the treatment comprising administering an effective dosage of testosterone and/or a functional analogue thereof to thereby treat said subject suffering from sexual dysfunction, and administering an effective dosage of oxytocin and/or a functional analogue thereof.

Oxytocin is a nonapeptide and a mammalian hormone. It is normally degraded in the gastrointestinal tract, therefore it is most commonly administered as liquid formulation by injection or as nasal spray. Synthetic oxytocin is commercially available as ready-to-use liquid formulations. “Functional analogues” of oxytocin are also known in the art, such as desamino-oxytocin (Hope et al., J Biol Chem 237 (5): 1563-1566, 1962) and carbetocin (Sweeney et al, Curr Ther Res 47:528-540, 1990), which are also commercially available. Hence, it is understood that a functional analogue of oxytocin includes any useful metabolite or precursor of oxytocin that can provide the same function as oxytocin.

In accordance with the invention, testosterone and oxytocin are provided and are found to be highly useful for the treatment of sexual dysfunction. It is understood functional equivalents of testosterone and oxytocin may be provided as well in one embodiment. And that in yet a further embodiment a combination of testosterone and a functional analogue thereof can be provided in accordance with the invention, or, oxytocin and a functional analogue thereof.

In the present invention, the use of testosterone and/or oxytocin, more preferably testosterone and oxytocin, was found to be highly useful in the treatment of sexual dysfunction as described herein. Sexual dysfunction includes symptoms such as a delayed, or loss of, sexual desire and/or arousal. Sexual arousal causes different physical changes, most significantly in the sex organs.

Sexual arousal in men, as a measure of sexual satisfaction, is preferably determined by determining an increase in size of penis and testes due to blood flow during an initial phase, and presence or absence of an orgasm and/or ejaculation in a next phase. The determination of presence of an orgasm and/or ejaculation may include determining the intensity of the orgasm and/or ejaculation.

Excitement in general can be triggered by thoughts, images, touch, scents, or any number of stimuli. Physiological signs of arousal include muscle tension, increased heart rate and breathing, elevated blood pressure, flushed skin, hardened or erect nipples, and blood flow to the genitals and pelvic region. Distraction, anxiety, stress, and depression can impact erection and arousal. Said excitement in male may cause a pre-ejaculate to present at the opening of the urethra. Continuous excitement may result in an orgasm and ejaculation, after which the body returns to an unexcited state.

Sexual desire/arousal in women, as a measure of sexual satisfaction, is preferably determined by determining vaginal lubrication in anticipation of sexual intercourse. Further indicators of sexual arousal in females are erection of nipples, vasocongestion of the vaginal walls, tumescence and erection of the clitoris and labia, elevation of the cervix and uterus, and expansion of the back of the vagina, a change in shape, color and size of the labia majora and labia minora, and/or pupil dilation. Sexual desire, or libido, is a subjective awareness of desire for sexual satisfaction, irrespective of sexual activity. Sexual desire is preferably determined by evaluation of an individual before and after administration in accordance with the invention of testosterone and/or a functional analogue, and oxytocin and/or a functional analogue thereof. The evaluation preferably relates to the individuals impression of improvement, to the number of sexually satisfying events, and to the levels of desire before and after the treatment. A preferred evaluation is provided by a questionnaire and/or a Sexual Event Diary (SED). Said SED is preferably completed at home within 24 hours of each sexual event. The SED measures different aspects of sexual satisfaction of a single sexual event (sexual desire, mental arousal, physical arousal, sexual pleasure, level of distraction, level of ability to let go, orgasm, satisfaction) using Likert-type scale items and binary questions. SED scores are preferably summarized per individual per regime, but only for those sexual events during which medication was used.

In a further embodiment, in accordance with the invention, subjects suffering from sexual dysfunction are selected from the group consisting of Female Sexual Interest/Arousal Disorder (FSIAD), Female Orgasmic Disorder, Hypoactive Sexual Desire Disorder (HSDD), Male Orgasmic Disorder and erectile dysfunction in males. In embodiment subjects suffer from the sexual dysfunction Female Sexual Interest/Arousal Disorder (FSIAD). In another embodiment from Female Orgasmic Disorder. In yet another embodiment, subjects suffer from Hypoactive Sexual Desire Disorder (HSDD). In still another embodiment, in accordance with the invention subjects suffer from Male Orgasmic Disorder. In another embodiment, subjects suffer from erectile dysfunction in males.

It is understood that the use of testosterone and/or a functional analogue thereof, and, oxytocin and/or a functional analogue thereof, are preferably for an on-demand use. An on-demand use means that the use involves administration prior to anticipated or planned sexual activity. A compound such as testosterone and/or a functional analogue thereof, and oxytocin and/or a functional analogue thereof, is preferably provided by a route of administration that is not invasive. Motivation for sexual behavior should normally not be negatively influenced by invasive routes of administration.

A steroid such as testosterone and/or a functional analogue thereof, is preferably provided by a route of administration, such as oral, buccal or intranasal administration, or by inhalation, for example by employment of a nebulizer. A preferred route of administration of a testosterone and/or a functional analogue thereof is oral administration, for example by a administering liquid formulation or by a sublingual formulation. Said sublingual formulation preferably comprises a displacement enhancer such as, for example, a cyclodextrin. Oral administration of testosterone is a comfortable option with minimal side effects such as accidental transfer of testosterone to women or children that can occur from transdermal products. In addition, oral administration of testosterone may increase adherence to treatment.

Said testosterone and/or a functional analogue thereof is preferably provided such that a short, sharp peak level of testosterone is present about 10-30 minutes, after administration of the testosterone and/or a functional analogue thereof.

Said short, sharp peak level of testosterone preferably is provided as a short, sharp peak level of free testosterone in the blood circulation of the human. Methods for determining the amount of free testosterone are known in the art such as, for example, described in Lavoie et al., 1989 (Lavoie et al., 1989. Clinical Biochemistry 22:451-456).

Testosterone is taught to increase the brain's sensitivity to cues associated with behavior driven by social interaction. In sexually functional women, the administration of 0.5 mg sublingual testosterone caused, after about three to four hours, an increase in brain sensitivity to sexual cues, which in turn affects their physiological sexual response in preparation for sexual behaviour (Tuiten et al., 2000. Arch Gen Psychiatry 57:149-53; Tuiten et al., 2002. Arch Gen Psychiatry 59:465). It is understood that the uses and methods as provided herein highly preferably include human subjects.

Without being bound by theory, administration of testosterone in accordance with the invention above a certain threshold value causes a pharmacological peak in testosterone. Above that threshold, administration of testosterone is accompanied by an increase in free testosterone. Free testosterone can have a biological and therefore a behavioral effect. An increase in testosterone does not have an immediate effect on certain stimulus-response relationships. An increase in free testosterone in the circulation is associated approximately 3-6 hours later with a greater sensitivity of brain areas to certain stimuli-response effects (it is about stimuli that are sensitive to testosterone, such as dominance, sexual behavior, and may include all kinds of cognitive functions). Testosterone administered e.g. sublingually with cyclodextrins (for women about 0.1-2 mg; for men about 0.5-20 mg) causes a pharmacological peak in testosterone after about 15 minutes. Hence, such a pharmacological peak e.g. induced by sublingual testosterone, can cause, e.g. in a period of 3 to 6 hours after administration, a greater sensitivity of the brain to sexual stimuli and therefore, an increasing effect on sexual motivation during this behavioral window.

In accordance with the invention, testosterone and/or a functional analogue thereof may preferably comprise testosterone and/or dihydrotestosterone. It is contemplated in accordance with the invention to use a mixture of testosterone and dihydrotestosterone. A mixture comprising testosterone and dihydrotestosterone preferably comprises said testosterone and dihydrotestosterone in a ratio of between 10:90 and 90:10 (w/w). Said ratio preferably is between 20:80 and 80:20 (w/w), more preferably between 30:70 and 70:30 (w/w), such as between 40:60 and 60:40 (w/w), including about 50:50 and 90:10 (w/w). A more preferred mixture may comprise testosterone and dihydrotestosterone in a ratio of between 10:90 and 90:10 (w/w). Said ratio preferably is between 25:75, about 30:70, such as about 40:60 and 45:55 (w/w).

In one embodiment, the total amount of testosterone and/or a functional analogue thereof, such as dihydrotestosterone, that is provided to an individual in need thereof is between 0.1 and 20 milligram. When the individual is a male, said testosterone and/or a functional analogue thereof, such as dihydrotestosterone, is preferably administered in a total amount of between 0.5 and 20 milligram, preferably between 1 and 10 milligram, including between 5 and 10 milligram. When the individual is a female, said testosterone and/or a functional analogue thereof, such as dihydrotestosterone, is preferably administered in a total amount of between 0.1 and 2 milligram, preferably between 0.25 and 1 milligram, such as between 0.3 and 0.7 milligram, including about 0.4 milligram, about 0.5 milligram and about 0.6 milligram.

In one embodiment, the total amount of testosterone and dihydrotestosterone, that is provided to an individual in need thereof is between 0.1 and 20 milligram. When the individual is a male, said testosterone and dihydrotestosterone, is preferably administered in a total amount of between 0.5 and 20 milligram, preferably between 1 and 10 milligram, including between 5 and 10 milligram. When the individual is a female, said testosterone and dihydrotestosterone, is preferably administered in a total amount of between 0.1 and 2 milligram, preferably between 0.25 and 1 milligram, such as between 0.3 and 0.7 milligram, including about 0.4 milligram, about 0.5 milligram and about 0.6 milligram. As shown in the examples herein, a suitable dosage of testosterone and dihydrotestosterone may be about 5 mg testosterone and about 5 mg dihydrotestosterone in male individuals, and about 0.5 mg testosterone and about 0.5 mg dihydrotestosterone in female individuals.

A preferred route of administration of testosterone and/or a functional analogue thereof, such as dihydrotestosterone, is oral administration, preferably by sublingual administration in the form of a sublingual formulation.

Said sublingual formulation is preferably provided as an immediate release delivery system for oral administration that is formulated to provide a peak level of free testosterone of at least 0,010 nmol/l in the blood circulation.

Accordingly, in another embodiment, testosterone and/or a functional analogue thereof, for use in accordance with the invention, is formulated to provide upon administration a peak level of free testosterone of 0,020 nmol/L or more for females, and 0,4 nmol/L or more for males in the blood circulation of the subject. In yet another embodiment, testosterone and/or a functional analogue thereof, for use in accordance with the invention, is formulated to provide upon administration a peak level of free testosterone of 0,020 nmol/L for females, and 0,4 nmol/L for males in the blood circulation of the subject. A preferred immediate release delivery system for oral administration comprises a core and a coating surrounding the core, said coating comprising said testosterone and and/or a functional analogue thereof, such as dihydrotestosterone, and further comprising a cyclodextrin. Said coating further preferably comprises a cellulose such as agglomerated cellulose, microcrystalline cellulose or a combination thereof. Said cellulose preferably is selected from methylcellulose, hydroxypropyl methylcellulose, and carboxy methyl cellulose, preferably is or comprises hydroxypropyl methylcellulose. The presence of a poorly soluble steroid such as testosterone and/or a functional analogue thereof, such as dihydrotestosterone, and a carrier such as a cyclodextrin provides rapid and efficient delivery of the testosterone and/or a functional analogue thereof, such as dihydrotestosterone, to the mucous membrane, from which the steroid is than rapidly absorbed into the circulation.

The neuropeptide oxytocin is understood to have a key role in the regulation of complex social cognitions and behaviors, such as attachment, parental care, pair bonding, as well as social exploration and recognition. It seems to play an important role in connecting social contacts with feelings of pleasure. It plays a central role in maternal bonding, friendships, and romantic interactions, as well as sexuality. The hormone is involved in many aspects of reproduction, including sexual behavior. Oxytocin can affect sexual arousal, orgasm, sexual satiety, and other aspects of social-sexual interactions. Intranasal administration of oxytocin causes an increase in oxytocin levels in the blood circulation within a few minutes, with the T-max occurring after approximately 15 minutes and returns to baseline after approximately 90 minutes. Means and methods to determine oxytocin levels in the blood are known in the art. Most studies that have investigated oxytocin having effect on behavior use dosages between 20-50 IE of oxytocin, most of them use 24 IE. Behavioral and neural effects of administered dosages are around 15-90 minutes after administration.

There is a time lag for the effect on the brain's sensitivity to cues associated with behavior of the testosterone and/or functional analogue thereof, of about 2.5-6 hours, which may be more specifically around 3-4.5 hours, in particular around 4 hours after (sublingual) administration. Furthermore, there is time lag for the effect on the brain's sensitivity to cues associated with behavior of the oxytocin and/or a functional analogue thereof, which is around 15-90 minutes after (intranasal) administration. Hence, testosterone and oxytocin, and the like, preferably is not administered at the same time and sublingual administration of testosterone and/or a functional analogue thereof, is preferably performed first, and, subsequently, secondly, oxytocin is administered, intranasally, in such a dosing regimen such that the sensitivity windows for testosterone (e.g. from about 2.5-6 hours after sublingual administration) and oxytocin (e.g. from about 15 minutes to 90 minutes after intranasal administration) overlap.

Hence, in one embodiment, administration of sublingual testosterone and/or a functional analogue thereof is followed by administering of intranasal oxytocin and/or a functional analogue thereof during a time window of about 3-6 hours after the peak concentration of testosterone in the blood circulation. Intranasal oxytocin may be preferably administered about 3.5 hours after the peak of testosterone, this way, the increased value of oxytocin associated with cognitive or other effects coincides with the period of highest behavioral effect of testosterone. It may preferred to initiate sexual activity after administration of oxytocin, preferably within 2 hours of administration of oxytocin, e.g. more preferably within 15-90 minutes after (intranasal) administration of oxytocin, such as within about 1 hours of administration of oxytocin. It may be preferred to have sexual activity after administration of oxytocin, preferably within 2 hours of administration of oxytocin, e.g. more preferably within 15-90 minutes after (intranasal) administration of oxytocin, such as within about 1 hours of administration of oxytocin. Hence, in another embodiment, any which way of administration selected for both testosterone and oxytocin, the administration of testosterone and/or a functional analogue thereof is to first provide a peak concentration of testosterone in the blood circulation, and second, the administration of oxytocin and/or a functional analogue thereof, is to provide a peak concentration of oxytocin and/or a functional analogue thereof, said peak concentrations separated from each other by about 3-4 hours. It is understood that sexual interaction may be initiated immediately once peak concentration of oxytocin and/or a functional analogue thereof, is obtained. In case of intranasal administration of oxytocin or the like, this may be initiated immediately, or shortly (e.g. within 15 minutes, or less) after administration. More preferably, sexual interaction is initiated in the time period between 15 minutes to 90 minutes after administration. Preferably, sexual interaction is engaged within 15 min-90 min after intranasal oxytocin administration.

As said, oxytocin is currently registered as a nasal spray, e.g. with a dose of 4 IE per spray and in containers carrying 50 doses. This is the preferred route of administration as it is relatively convenient. Nevertheless, further suitable forms of oxytocin and/or further suitable means may be contemplated in accordance with the invention that can provide for an effect as observed with intranasally administered oxytocin and that may be suitable for the highly advantageous uses and methods in accordance with the invention, i.e. provide for likewise behavioral and neural effects as observed with intranasal administration. One may even contemplate intravenous administration. Highly advantageously, it may also be contemplated to administer oxytocin in a limited sustained release form, sublingual form, intramuscular injection, inhalation, or through a hydrogel. Although such alternative means of administration may currently not be registered, such forms have been developed and such forms may provide highly useful alternative means of administration of oxytocin.

Accordingly, in one embodiment, testosterone and/or a functional analogue thereof for use in accordance with in the invention is provided, wherein the treatment of sexual dysfunction further comprises administering an effective dosage of oxytocin and/or a functional analogue thereof, wherein said effective dosage of oxytocin and/or a functional analogue thereof is administered in a form to provide increased oxytocin levels 4-5 hours after the peak level in the blood level of the subject of the testosterone and/or the functional analogue.

In yet another embodiment, testosterone and/or a functional analogue thereof for use in accordance with in the invention is provided, wherein the treatment of sexual dysfunction further comprises administering an effective dosage of oxytocin and/or a functional analogue thereof, wherein said effective dosage of oxytocin and/or a functional analogue thereof is administered in a form to provide increased oxytocin levels preferably 3-4 hours, or about 3.5 hours, after the peak level of free testosterone.

Preferably, in accordance with the invention, treatments comprising an effective dosage of oxytocin and/or a functional analogue thereof, comprise intranasal administration of said effective dosage of oxytocin and/or a functional analogue thereof.

In yet another embodiment, treatments in accordance with the invention comprising administering an effective dosage of oxytocin and/or a functional analogue thereof, include administration of a total amount in the range of 10-100 IE, preferably in the range of 20-50 IE. It is understood that such amounts preferably relate to intranasally administered amounts of oxytocin and/or a functional analogue, and alternative administration routes may involve equivalent amounts. A suitable amount for administration that may be selected is 24 IU.

Suitable amounts for administration in accordance with the invention that may be selected include about 24 IE of ocytocin, and about 5 mg of testosterone for a male subject, and about 0.5 mg of testosterone for a female subject. Suitable amounts for administration in accordance with the invention that may be selected include 24 IE of ocytocin, and 5 mg of testosterone for a male subject, and 0.5 mg of testosterone for a female subject, such as shown in the example section herein, as described herein.

In another embodiment, a method of treatment is provided of a subject suffering from sexual dysfunction, the treatment comprising:
administering an effective dosage of testosterone and/or a functional analogue thereof, as defined herein;
administering an effective dosage of oxytocin and/or a functional analogue thereof, as defined herein.

In yet another embodiment, a method of treatment of a subject suffering from sexual dysfunction is provided, the treatment comprising:
administering an effective dosage of testosterone and/or a functional analogue thereof, sublingually, in an immediate release form, and,
administering an effective dosage of oxytocin and/or afunctional analogue thereof, intranasally, about 3-5 hours, preferably 3, 5 hours, after administering the testosterone and/or the functional analogue thereof.

In yet another further embodiment, a method of treatment of a subject suffering from sexual dysfunction is provided, the treatment comprising:
administering an effective dosage of testosterone and/or a functional analogue thereof, sublingually, in an immediate release form, and,
administering an effective dosage of oxytocin and/or a functional analogue thereof, intranasally, about 3-5 hours, preferably 3, 5 hours after administering the testosterone and/or the functional analogue thereof, and
wherein sexual activity may be initiated within 2 hours after intranasal administration of oxytocin.

In yet another further embodiment, a method of treatment of a subject suffering from sexual dysfunction is provided, the treatment comprising:
administering an effective dosage of testosterone and/or a functional analogue thereof, sublingually, in an immediate release form, and,
administering an effective dosage of oxytocin and/or a functional analogue thereof, intranasally, about 3-5 hours, preferably 3, 5 hours after administering the testosterone and/or the functional analogue thereof, and
wherein sexual activity may be initiated 15-90 minutes after intranasal administration of oxytocin.

In yet another further embodiment, a method of treatment of a subject suffering from sexual dysfunction is provided, the treatment comprising:
administering an effective dosage of testosterone and dihydrotestosterone, sublingually, in an immediate release form, and,
administering an effective dosage of oxytocin and/or a functional analogue thereof, intranasally, about 3-5 hours, preferably 3, 5 hours after administering the testosterone and/or dihydrotestosterone, and
wherein sexual activity may be initiated 15-90 minutes after intranasal administration of oxytocin.

In still another further embodiment, a method of treatment of a subject suffering from sexual dysfunction is provided, the treatment comprising:
administering an effective dosage of testosterone and dihydrotestosterone, sublingually, in an immediate release form, and,
administering an effective dosage of oxytocin thereof, intranasally, about 3-5 hours, preferably 3, 5 hours after administering the testosterone and dihydrotestosterone, and
wherein sexual activity may be initiated 15-90 minutes after intranasal administration of oxytocin.

Furthermore, the invention provides for oxytocin and/or a functional analogue thereof for use in a treatment of a subject suffering from sexual dysfunction, the treatment comprising administering an effective dosage of oxytocin and/or a functional analogue thereof as defined herein to thereby treat said subject suffering from sexual dysfunction.

In a further embodiment, the invention provides for oxytocin and/or a functional analogue for use in the treatment of a subject suffering from sexual dysfunction, the treatment comprising:
administering an effective dosage of testosterone and/or a functional analogue thereof, as defined herein;
administering an effective dosage of oxytocin and/or a functional analogue thereof, as defined herein.

In yet another embodiment, the invention provides for testosterone and/or a functional analogue thereof, and, oxytocin and/or a functional analogue thereof, for use in a treatment of a subject suffering from sexual dysfunction as defined herein.

Sexual performance, when subjecting subjects to treatments as contemplated herein, such as a treatment comprising a combination of testosterone and oxytocin, have improved sexual arousal during sexual activity, an improved orgasm, improved horniness preceding sexual activity, improved horniness during sexual activity, or a reduction in the time before an orgasm as compared with not receiving such a treatment. Sexual performance, when subjecting subjects to treatments as contemplated herein, such as a treatment comprising a combination of testosterone and oxytocin, may have one or more of the following improved, sexual arousal during sexual activity, orgasm, horniness preceding sexual activity, horniness during sexual activity, as compared with not receiving such a treatment.

### Testosterone, Dihydrotestosterone and a PDE5 Inhibitor

As shown in the examples herein, in an alternative embodiment, testosterone and dihydrotestosterone, was also shown, when combined with Viagra, i.e. sildenafil, a PDE5 inhibitor, to provide for a highly improved effect over sexual arousal over the testosterone combined with Viagra. A phosphodiesterase type 5 inhibitor (PDE5 inhibitor) is a vasodilating drug that works by blocking the degradative action of cGMP-specific phosphodiesterase type 5 (PDE5) on cyclic GMP in the smooth muscle cells lining the blood vessels supplying various tissues. Hence one such embodiment, also provided herein, is the combination of testosterone and dihydrotestosterone with a PDE5 inhibitor for the treatment of sexual dysfunction. Further PDE5 inhibitors are known in the art, and can be selected from vardenafil, sildenafil, tadalafil and other PDE5 inhibitors. A PDE5 inhibitor may be selected from any of vardenafil, sildenafil, tadalafil and any other suitable PDE5-inhibitor.

The combination of testosterone and dihydrotestosterone and a PDE5 inhibitor, in the alternative embodiments as contemplated herein, may be selected and used in a treatment similar to the combination of testosterone and/or dihydrotestosterone and oxytocin, as contemplated herein above. Dosages of testosterone and/or dihydrotestosterone as described herein for male and female subjects may apply in this alternative embodiment, as well as means of administrations.

With regard to timing of administration of testosterone and dihydrotestosterone prior to the administration of the PDE5 inhibitor, this may be such that is provided such that after co-administration of testosterone and dihydrotestosterone, the peak plasma level of testosterone and free testosterone occurs about 3-6 hours, more preferred 4-5 hours, prior to the peak plasma level of the PDE5 inhibitor. For example, by administration of an oral formulation of testosterone and dihydrotestosterone such as described in the examples herein, followed by administration of about 2.5 hours later of Viagra (an available formulation of sildenafil), after which, subjects are engaged in sexual activity 1-1.5 hour hours later. Suitable dosage forms for the combined delivery of testosterone and PDE5 inhibitors are known in the art (WO2017204632, WO2012158030), which is herein incorporated by reference, and such dosage forms can easily be adapted in accordance with this alternative embodiment as presented herein with the advantageous combination of PDE5 inhibitor, testosterone and dihydrotestosterone, by replacing the dosage form of testosterone as described therein, with the combination of testosterone and dihydrotestosterone as contemplated herein instead.

Suitable dosage forms for the combined immediate release delivery of testosterone and/or dihydrotestosterone as already described herein above, may also be contemplated combined with known sustained release dosage forms PDE5 inhibitors, i.e. pills comprising PDE5 inhibitors, e.g. by coating dosage forms of a PDE 5 inhibitor with an immediate release coating comprising testosterone and dihydrotestosterone. Of course, taking into account the peak plasma levels and/or timing of administration as described herein, which may require adaptation e.g. of sustained release formulation.

For these alternative embodiments, suitable combinations of testosterone, dihydrotestosterone and a PDE5 inhibitor, may comprise the testosterone and dihydrotestosterone dosages as contemplated herein throughout, as contemplated for the combination of testosterone and/or functional analogue, such as dihydrotestosterone, and, optionally, oxytocin, as contemplated herein for male and female subjects suffering from sexual dysfunctions. Such combinations of testosterone, dihydrotestosterone and a PDE5 inhibitor, in these alternative embodiments, comprise suitable and appropriate dosages of a PDE5 inhibitor for male and female subjects suffering from sexual dysfunction, such as 50 mg of sildenafil for a male patient, combined with e.g. 5 mg of testosterone and 5 mg of dihydrotestosterone, as described in the examples herein. Combinations of testosterone, dihydrotestosterone and a PDE5 inhibitor, for female subjects suffering from sexual dysfunction may comprise suitable and appropriate dosages of a PDE5 inhibitor, such as 50 mg of sildenafil combined with e.g. 0.5 mg of testosterone and 0.5 mg of dihydrotestosterone.

### Examples

Testosterone and Dihydrotestosterone Combined with a PDE5 Inhibitor Improves Sexual Function

The first example concerns two male heterosexual patients (64 and 67 years) with sexual problems (erection problems and reduced sexual arousal). The usual treatments (such as Viagra and Androgel) have had little or no effect on them. This prompted two alternative forms of treatment. The first treatment involved taking 5 mg sublingual testosterone (in a solution consisting of alcohol, denaturalized water, testosterone and cyclodextrins) followed 2.5 hours later by taking Viagra (sildenafil 50 mg). The second treatment consisted of simultaneously taking 5 mg sublingual testosterone and 5 mg sublingual dihydrotestosterone (DHT) (in a solution consisting of alcohol, denaturalized water, DHT and cyclodextrins), followed after 2.5 hours by taking Viagra (50 mg). The testosterone and DHT solutions were prepared by a pharmacist on the request of a general practitioner. On both treatment days, patients were asked to engage in sexual activity between 1 and 1.5 hours after taking Viagra. Subsequently, the changes in total testosterone and free testosterone in blood plasma were examined, 15 minutes after intake of the androgen(s). Patients were also asked to answer some questions about their sexual arousal immediately before and within an hour after sexual activity.

Questions: On a scale of 1 to 10 (from not at all to very much), would you rate your level of sexual arousal a) before and b) during sexual activity? Please circle: Not at all [1−[ . . . ]−10] Very much

The table below describes the effects of the different treatments on ‘total testosterone’ and ‘free testosterone (i.e. measured, not calculated)’ for both patients:

| | | | | Total T | Free T | SHBG | PSA |
|---|---|---|---|---|---|---|---|
| | | DOT | date | nmol/L | pml/L | nmol/L | μmol/L |
| Patient 1 | No Drug | 1 | 7-3 | 10.7 | 190 | 35 | 0.60 |
| | T | 1 | 7-3 | 28.7 | 440 | | |
| | T + DHT | 2 | 10-3 | 28.8 | 560 | | |
| Patient 2 | No Drug | 1 | 7-3 | 11.1 | 220 | 29 | 1.80 |
| | Testosterone (T) | 1 | 7-3 | 43.9 | 1030 | | |
| | T + DHT | 2 | 10-3 | 44.2 | 1096 | | |

T = testosterone
DHT = dihydrotestosterone

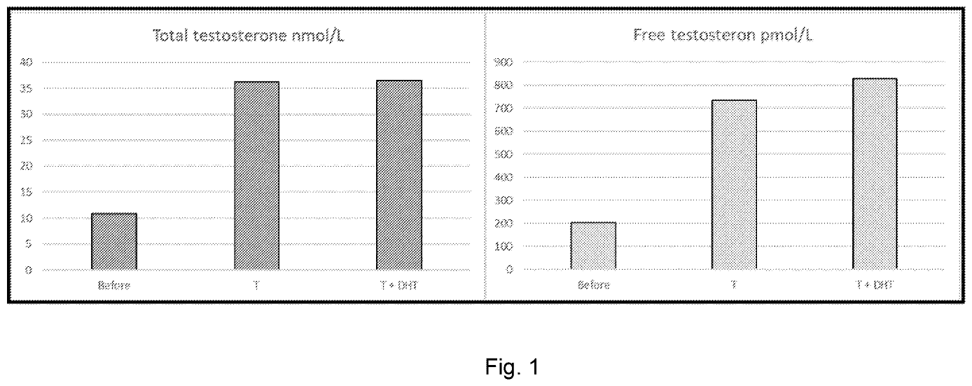

These results are summarized in FIG. 1, depicting the average of the two patients.

The table and FIG. 1 shows that sublingual T and sublingual T & DHT have an equal effect on total testosterone (FIG. 1, left figure), while sublingual T+DHT (compared to T alone) appears to have a greater effect on free testosterone (FIG. 1, to the right). This is consistent with the assumption that SHBG becomes more saturated as a result of the binding of DHT to SHBG. This means that the ingested testosterone can no longer bind to SHBG, and consequently there will be a greater increase in unbound testosterone (i.e. free testosterone). By combining sublingual T with DHT, free testosterone is higher compared to sublingual T alone (as stated above). Consequently, by a combination of T and DHT a lower dosage of sublingual T can be used to reach the same level of free testosterone as compared to sublingual T alone. Total testosterone and free testosterone were measured 15 minutes after the administration of T and (T&DHT).

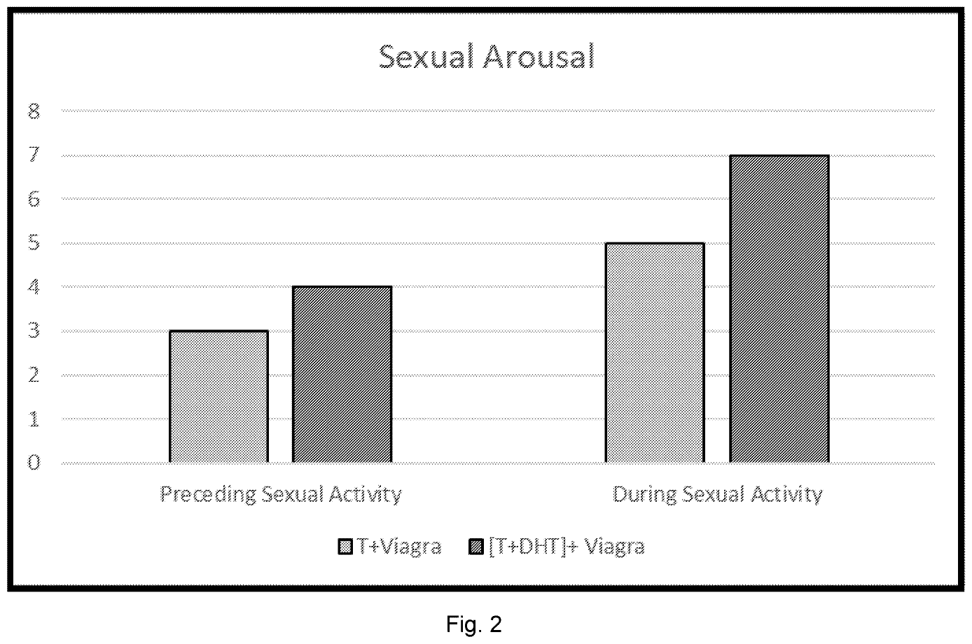

There also appear to be differences in sexual arousal during the different androgen treatments both immediately before and during sexual activity (measured approximately 1 to 1.5 hours after sexual activity). Both before and after sexual activity, administration of T+DHT (and Viagra) is associated with greater sexual arousal compared to T and Viagra alone. See FIG. 2.

These results show a greater influence of T+DHT (and Viagra) on free testosterone and on sexual arousal than T (and Viagra) alone. This effect on sexual arousal may be due to more free testosterone in the T+DHT (and Viagra) condition, or due to a yet unidentified interaction between T and DHT on sexual arousal.

The mechanisms discussed differ little between men and women, except with regard to a difference in male and female regarding dosages of testosterone and dihydrotestosterone. It is expected that the use of 0.5 mg sublingual testosterone combined with 0.5 mg sublingual dihydrotestosterone in women will also lead to an increase in free testosterone, and in combination with Viagra (50 mg), to an improvement in sexual symptoms.

### a Combination of Testosterone and Oxytocin Improves Sexual Function

A key finding in the present disclosure with regard to sexual dysfunctions in women and men is that sublingual T (5 mg for men and 0.5 mg for women) combined with administration of 24 units (IU) of intranasal oxytocin 3.5 hours after the peak in free testosterone in plasma, as expected from known pharmacokinetic properties of testosterone, results in an improvement in sexual functioning.

Two patients (one woman and one man) with sexual complaints (low sex drive and reduced sexual arousal) were treated once with this combination (with informed consent). Patients were instructed to initiate and/or engage sexual activity 15-90 minutes after oxytocin administration. Both were asked to answer some questions regarding their sexual functioning, on a scale of 1 to 10 (once without medication and once with medication):

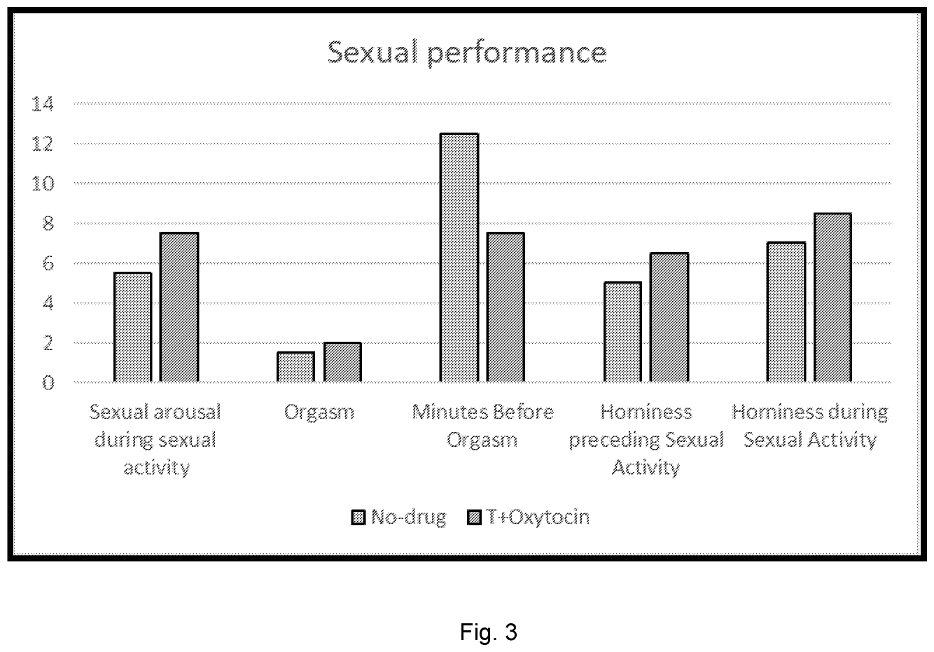

Questionnaires were completed after sexual activity. The average results of both subjects can be found in FIG. 3.

Based on the results, administration of sublingual testosterone, followed by administration of 24IU oxytocin 3.5 hours after the peak in testosterone is associated with an improvement in a number of aspects of sexual functioning in these patients, as compared with no medication.

As shown in the treatment examples described herein above, described are various improved aspects of sexual functioning of patients suffering from sexual dysfunctions. Likewise, patients suffering from sexual dysfunctions are treated with a combination of testosterone, dihydrotestosterone and oxytocin, for improvement of sexual functioning. Female subjects are treated with 0.5 mg sublingual testosterone combined with 0.5 mg sublingual dihydrotestosterone and 24 IU oxytocin in a regimen as described above for the combination of testosterone and oxytocin, whereas male subjects are treated with 5 mg sublingual testosterone combined with 5 mg sublingual dihydrotestosterone.

1. A method of treatment of a subject suffering from sexual dysfunction, the treatment comprising
administering an effective dosage of testosterone and/or a functional analogue thereof to thereby treat said subject suffering from sexual dysfunction.

2. The method of claim 1, further comprising
administering an effective dosage of oxytocin and/or a functional analogue thereof.

3. The method of claim 1, wherein the subject suffers from a sexual dysfunction selected from the group consisting of
Female Sexual Interest/Arousal Disorder (FSIAD), Female Orgasmic Disorder, Hypoactive Sexual Desire Disorder (HSDD), Male Orgasmic Disorder and erectile dysfunction in males.

4. The method of claim 1, wherein administering to the subject suffering from sexual dysfunction occurs prior to anticipated or planned sexual activity.

5. The method of claim 1, wherein testosterone and/or a functional analogue is administered in a total amount in the range of 0.1-20 mg.

6. The method of claim 1, wherein the subject is a female subject and wherein said testosterone and/or a functional analogue is administered in a total amount in the range of 0.1-2 mg, or, wherein the subject is a male subject and wherein said testosterone and/or a functional analogue is administered in a total amount in the range of 0.5-20 mg.

7. (canceled)

8. The method of claim 1, whereby the testosterone and/or the functional analogue thereof is formulated to provide upon administration a peak level of free testosterone of 0.020 nmol/L or more for females, and 0.4 nmol/L for males in the blood circulation of the subject.

9. The method of claim 1, wherein the subject is a human.

10. The method of claim 1, wherein the functional analogue of testosterone is dihydrotestosterone.

11. The method of claim 1, wherein said testosterone and/or a functional analogue thereof is provided in the form of a sublingual formulation.

12. The method of claim 1, wherein the testosterone and/or a functional analogue thereof is in the form of an immediate release formulation for oral on-demand administration.

13. The method of claim 12, wherein the immediate release formulation comprises a coating, said coating comprising the testosterone and/or the functional analogue thereof, and further comprises a cyclodextrin, or a derivative or polymer thereof.

14. The method of claim 2, wherein said effective dosage of oxytocin and/or a functional analogue thereof is administered in a form to provide increased oxytocin levels 3-5 hours after the peak level in the blood circulation of the subject of the testosterone and/or the functional analogue thereof.

15. The method of claim 2, wherein said effective dosage of oxytocin and/or a functional analogue thereof is administered intranasally.

16. The method of claim 15, wherein said effective dosage of oxytocin and/or a functional analogue thereof is administered intranasally about 3-5 hours after administering testosterone and/or a functional analogue thereof.

17. The method of claim 2, wherein said effective dosage of oxytocin and/or a functional analogue thereof is administered in a total amount in the range of 10-100 IE.

18. The method of claim 2, wherein the treatment further comprises administering an effective dosage of oxytocin and/or a functional analogue thereof, wherein sexual activity is initiated within 2 hours after intranasal administration of oxytocin.

19-20. (canceled)

21. A method of treatment of a subject suffering from sexual dysfunction, the treatment comprising
administering an effective dosage of oxytocin and/or a functional analogue thereof to thereby treat said subject suffering from sexual dysfunction.

22. The method of claim 21, further comprising:
administering an effective dosage of testosterone and/or a functional analogue thereof.

23. (canceled)

24. The method of claim 2, wherein the subject suffers from a sexual dysfunction selected from the group consisting of
Female Sexual Interest/Arousal Disorder (FSIAD), Female Orgasmic Disorder, Hypoactive Sexual Desire Disorder (HSDD), Male Orgasmic Disorder and erectile dysfunction in males.
